(19) United States
(12) Patent Application Publication
(10) Pub. No.: US 2004/0260392 A1
(43) Pub. Date: Dec. 23, 2004

(54) Arterial prosthesis

(76) Inventors: Viktoria Kantsevitcha, Riga (LV); Eriks Masteiko, Jurmala (LV); Leonids Ribickis, Riga (LV)

Correspondence Address: Colin P. Abrahams, Suite 400, 5850 Canoga Avenue, Woodland Hills, CA, 91367, US

(21) Appl. No.: 10/775,826

(22) Filed: Feb. 10, 2004

Related U.S. Application Data
(63) Continuation-in-part of application No. 10/204,009, filed on Oct. 10, 2002, now Pat. No. 6,709,467.

(30) Foreign Application Priority Data
Feb. 16, 2000 (LV) P-00-21

(51) Int. Cl. A61F 2/06

(52) U.S. Cl. 623/1.44; 623/1.53

(57) ABSTRACT

An arterial prosthesis comprising biological inert polyester and polyurethane yarns, at last of portion of said yarns being agglutinated with gelatin/glycerin solution bonds.

### CROSS REFERENCE TO RELATED APPLICATIONS

This application is a continuation-in-part application of U.S. patent application Ser. No. 10/204,009 filed Aug. 15, 2002, and which is incorporated herein in its entirety.

### FIELD AND BACKGROUND OF THE INVENTION

The present invention relates to a medical technique. It can be used in the reconstructive surgery in cases where the circulatory system has congenital anomalies or the subject suffers from atherosclerosis, injuries or any other detriment.

There exists a flexible blood vessel prosthesis (LV patent No. 12175) consisting of polyester and polyurethane yarns with a lining of velour type crimps on its walls. The said prosthesis represents the following disadvantages:

after implantation the structure of the prosthesis cannot prevent blood leakage through it;

the ends of the prosthesis ravel easily; it makes it difficult to suture the prosthesis to the natural blood vessel.

### SUMMARY OF THE INVENTION

One aspect of the present invention is to produce an arterial prosthesis that easily modulates when continuous blood flow is pumped through it at a definite pressure and speed. The prosthesis should substantially exclude blood leakage through its walls, and its ends should preferably be easily attachable to natural blood vessels.

### BRIEF DESCRIPTION OF THE DRAWINGS

FIG. 1 is a cross-section through an arterial prosthesis in accordance with one embodiment of the invention;

FIGS. 2 and 3 show measurements of strain and force and width and strain respectively; and

FIG. 4 is a graph showing the measurement of pressure against circumferential stretch ratio.

### DETAILED DESCRIPTION OF THE INVENTION

The arterial prosthesis is produced using weaving technology. In the weaving machine two warps of polyester yarns are arranged (the number of yarns corresponds to the one that ensures the required diameter of the tube), and two warps of polyurethane yarns. The weft consists of three-yarn systems (one polyester yarn and two polyurethane yarns). All polyurethane yarns are passed to the operational area at a 200% longitudinal stretch. A continuous tube is woven in a complicated braided pattern (two-layered). In each section (see FIG. 1) the cop lays four polyurethane (1) and two polyester (3) yarns, three yarns—from the left towards the right, and three yarns—when returning to the same section from the right to the left, fixing the first three weft yarns on the reed beforehand. The laid weft yarns get compressed between tensioned polyurethane warps (2) and form the intraluminal coat of the prosthesis. The outer surface is formed by polyester warp yarns (4), that lay in a crimpy velour type structure beyond the operational area of the weaving machine when the polyurethane yarns relax.

The arterial prosthesis produced by the said technique, ensures a continuous blood flow; it easily modulates both radially and longitudinally. The internal coat prevents blood from leaking through walls of the prosthesis after implantation, and the interbraiding of both layers form ends of prosthesis that ravel little. In order to enhance the above features and to ensure safety, the prosthesis gets thermostabilized and vacuum-impregnated with the solution of gelatin and glycerin. When drying up, the solution binds filaments of the polyester yarn and pores of the prosthesis, thus eliminating or reducing the permeability of the prosthesis, and its ends become easily attachable to the natural blood vessel (they do not ravel). Then implanted, the gelatin and glycerin bonds fill out and through them the natural tissue ingrows, thus forming a dense mesh of capillaries and a stable “neo-intime”.

Based on the knowledge of mechanical properties and structure of human arteries, the criteria for design of arterial grafts which match to the host artery is developed. An elastic pre-stretched polyurethane mono-filament thread with a low modulus of elasticity and a polyester multi-filament with a high modulus of elasticity are used. Technical parameters are determined and a composite vascular graft of diameter about 4 mm is constructed. Mechanical tests carried out indicate that the compliance of the vascular grafts were similar with that of the human carotid artery.

The replacement of small diameter arteries (such as the coronary, renal, carotid and long part of vessels in the legs) by grafts is a challenging issue in reconstructive surgery. One difficulty which has resulted in poor performance of such existing prostheses may be the lack of compliance. A replacement of small arteries by rigid prostheses may cause a formation of thrombus and hyperplastic intima.

A successful development of a small diameter vascular graft will depend not only on the use of biocompatible materials, but also on vascular graft construction. One aspect of the present invention relates to a non-linear compliant composite vascular graft. To minimize the degree of implantation risk, the invention in one aspect relates to a new structure of a composite compliant vascular graft. In one aspect of the invention, this structure is capable of being deformed in an axial direction up to 50% without changing diameter of the vascular graft, and in a circumferential direction of up to 10-12% at an internal pressure of 240 mmHg.

The vascular graft in accordance with one embodiment of the present invention may be developed using a complex interlacement from biologically compatible and neutral living tissues, a multi-filament polyester and mono-filament polyurethane thread. Preferably, the ratio of these components is 1:1 on a warp and on a weft. The polyester threads may carry out the role of collagen, and the polyurethane threads the role of elastin. The interlacement provides on the outer surface of the vascular graft a loop-shaped structure from the polyester threads, and on the internal part of the vascular graft there is formed sufficient smooth surface. The average part of such tubular vascular grafts is generated from a polyester weft clamped between pre-stretched polyurethane warp and weft. Such structure of a wall of the compliant vascular graft facilitates “implantation” of a capillary net and living tissues, and also provides the minimal opportunity of infiltration of blood through the walls immediately after implantation. Water permeability of the vascular grafts preferably does not exceed 0.15-0.20 l/min.cm2. Beside the vascular graft, after implantation, in a general stream of blood, flow begins to pulse at once.

In weaving technology, a very important factor is the refueling tension of polyurethane threads which depends not only on the structure of the wall of the vascular graft, but also its ability to be deformed in both the longitudinal and the circumferential directions. There has therefore been a study of the width A (mm) changing and absolute lengthening L (mm), and a relative strain S (%) of mono-filament polyurethane threads at various loads, all of which are of interest in the manufacture of composite compliant vascular grafts.

Experiments and Results

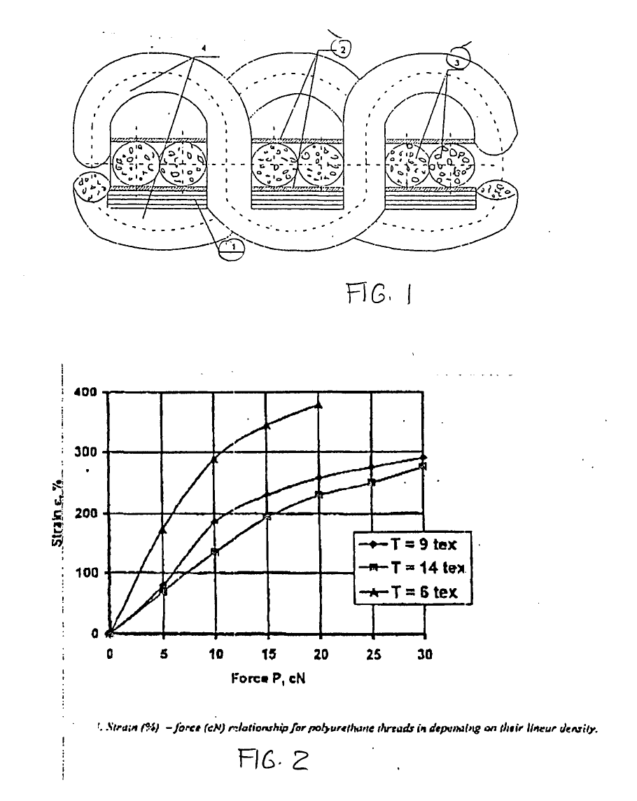

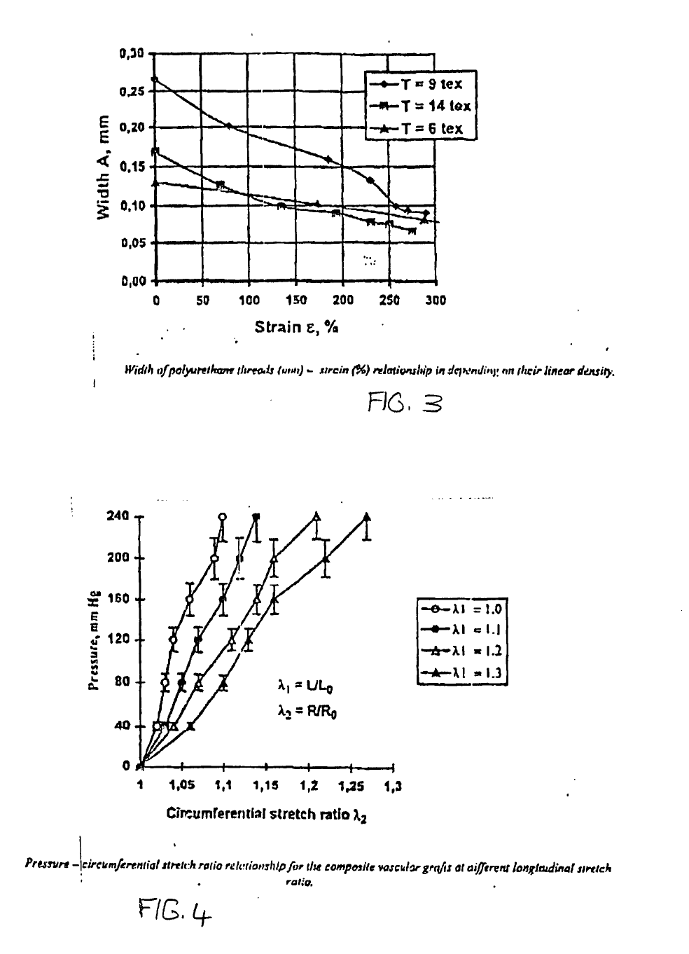

Experiments were carried out using polyurethane threads which were manufactured in Russia and in the U.S.A. Thirty-five bobbins of each version were checked. From each bobbin there were made five measurements (in the Table, average values are given). Results of these measurements are shown in Tables 1 to 3 below, and FIGS. 2 and 3. The analysis of experimental data shows that processing of mono-filament polyurethane threads with T=9.1 tex (Russia) and T=14 tex (U.S.A.) in a base on rapier weaving looms AR-1, the refueling tension may provide normal work at value F_{arrangement}=25cN/thread. Such tension reduces a width of thread in a working zone of the machine tool on ΔA÷0.55×0.64%, and relative strain of the thread will be about ε÷250×270%. Accordingly, in the use of the polyurethane threads T=6 tex, the refueling tension will be about F=10 cN/thread. This will reduce a width of a thread on about 51% and the relative strain of the thread will be about ε=288%.

TABLE 1
Characteristics of polyurethane threads

| P | Polyurethane threads: T = 9.1 tex (Russia) | | | |
|---|---|---|---|---|
| [cN] | A [mm] | L [mm] | ε [%] | ΔA [%] |
| 0 | 0.267 | 10.0 | 0 | 0 |
| 5 | 0.201 | 18.0 | 80 | −25 |
| 10 | 0.159 | 28.5 | 185 | −40 |
| 15 | 0.132 | 33.0 | 230 | −51 |
| 20 | 0.099 | 35.8 | 258 | −63 |
| 25 | 0.093 | 37.0 | 270 | −64 |
| 30 | 0.090 | 39.0 | 290 | −66 |

TABLE 2
Characteristics of polyurethane threads

| P | Polyurethane threads: T = 14 tex (USA) | | | |
|---|---|---|---|---|
| [cN] | A [mm] | L [mm] | ε [%] | ΔA [%] |
| 0 | 0.168 | 10.0 | 0 | 0 |
| 5 | 0.126 | 17.0 | 70 | −25 |
| 10 | 0.099 | 23.5 | 135 | −41 |
| 15 | 0.090 | 29.3 | 193 | −46 |
| 20 | 0.078 | 33.0 | 230 | −54 |
| 25 | 0.075 | 35.0 | 250 | −55 |
| 30 | 0.066 | 37.0 | 275 | −61 |

TABLE 3
Characteristics of polyurethane threads

| P | Polyurethane threads: T = 6 tex (USA) | | | |
|---|---|---|---|---|
| [cN] | A [mm] | L [mm] | ε [%] | ΔA [%] |
| 0 | 0.130 | 10.0 | 0 | 0 |
| 5 | 0.102 | 27.3 | 173 | −38 |
| 10 | 0.081 | 38.8 | 288 | −51 |
| 15 | 0.066 | 44.3 | 343 | −60 |
| 20 | 0.051 | 47.8 | 378 | −69 |
| 25 | break | break | break | break |

Use of this data in the manufacture of new structures of vascular grafts has provided a pure shred, a normal surf of a weft to a margin of a product. A changing of the thickness of the wall at various loadings of the polyurethane thread is shown in various models. After a breast beam, polyurethane threads of a warp become shorter due to relaxation, but keep the relative strain within the limits of about ε=100÷125%.

Polyurethane threads of the weft at the moment of a submission on a rapier should have a tension F=10 cN/thread, which, as a result of a rapier passing through a shred, is increased 2.5 times and at the moment of a surf, F=25 cN/thread. Reliability of experimental results is believed to be about 94-95%. Experimental results shown in FIG. 4 indicate that increasing of longitudinal stretch ratio of the vascular graft leads to increasing compliance in the circumferential direction. For example, at the internal pressure 120 mm Hg, the circumferential stretch ratio increases from about 1.04 (at the longitudinal stretch ratio 1.0) to about 1.13 (at the longitudinal stretch ratio 1.13). Prestretch of the vascular graft in the longitudinal direction during implantation will increase compliance of the graft.

1. An arterial prosthesis comprising biological inert polyester and polyurethane yarns, at last of portion of said yarns being agglutinated with gelatin/glycerin solution bonds.

2. An arterial prosthesis as claimed in claim 1 comprising four layers of polyurethane and two layers of polyester, the polyester being compressed between tensioned polyurethane walls.

3. An arterial prosthesis as claimed in claim 1 comprising a first woven layer interbraided with a second woven layer, the layers being comprised of a biologically inert polyester and polyurethane yarns, at last one coat of gelatin/glycerin bonds on the first and/or second woven layers.

4. A method of manufacturing an arterial prosthesis comprising interbraiding a first woven layer with a second woven layer, the layers being comprised of biologically inert polyester and polyurethane yarns, and applying internal and external coats of gelatin/glycerin solution bonds thereto.
